(19) United States
(12) Patent Application Publication
(10) Pub. No.: US 2025/0029684 A1
(43) Pub. Date: Jan. 23, 2025

(54) MICROSCOPIC COMPARISON AND EXPERIMENTAL VERIFICATION METHOD FOR WETTABILITY OF SIMILAR SURFACTANTS ON COAL DUST

(71) Applicant: SHANDONG UNIVERSITY OF SCIENCE AND TECHNOLOGY, QINGDAO (CN)

(72) Inventors: WEN NIE, QINGDAO (CN); WENJIN NIU, QINGDAO (CN); QIU BAO, QINGDAO (CN); QIFAN TIAN, QINGDAO (CN); RUOXI LI, QINGDAO (CN); HUAJUN LIU, QINGDAO (CN); JING YANG, QINGDAO (CN); XIAOHAN ZHANG, QINGDAO (CN); JIE LIAN, QINGDAO (CN); LIDIAN GUO, QINGDAO (CN); CHENGYI LIU, QINGDAO (CN); CHANGWEI XU, QINGDAO (CN)

(21) Appl. No.: 18/757,981

(22) Filed: Jun. 28, 2024

Related U.S. Application Data
(63) Continuation of application No. PCT/CN2023/138373, filed on Dec. 13, 2023.

(30) Foreign Application Priority Data
Jul. 20, 2023 (CN) 202310895022.8

(51) Int. Cl. G16C 20/30; G01N 21/3563; G01N 24/08; G16C 20/20

(52) U.S. Cl. CPC G16C 20/30; G01N 21/3563; G01N 24/081; G16C 20/20

(57) ABSTRACT

Disclosed is a microscopic comparison and experimental verification method for the wettability of similar surfactants on coal dust, comprising: collecting a coal sample on site from a mining area and analyzing the coal sample by infrared spectroscopy and nuclear magnetic resonance spectroscopy to obtain information on functional groups and carbon structure of coal molecules; S2: constructing a surfactant-coal molecule electrostatic interaction model and analyzing an orbital energy difference and an electrostatic potential difference between surfactant molecules and coal molecules, etc. S6. The invention provides a simple and effective idea for the selection of surfactants.

### BACKGROUND OF THE INVENTION

### 1. Technical Field

The present invention belongs to the technical field of coal dust prevention and control and particularly relates to a microscopic comparison and experimental verification method for the wettability of similar surfactants on coal dust.

### 2. Description of Related Art

Due to the hydrophobicity and suspension of coal dust, the dust suppression effect of water mist spraying is not ideal, and the existence of dust suppressants just makes up for this defect. However, raw materials for dust suppression are often selected by way of experiments. Once more than 20 or 30 kinds of raw materials are involved, the experiments will be laborious and difficult.

With the continuous innovation of science and technology, molecular dynamics simulation has become a powerful tool for understanding the structure, characteristics and reaction mechanism analysis of materials in various fields such as physics, chemistry, materials and biology. It can not only analyze the structure and related characteristics of molecules generating absorption, but also simulate the dynamic behavior of molecules during the adsorption process. It is another important method for exploration at the microscopic level in addition to theoretical and experimental researches. Therefore, combined with the molecular simulation technology, a method for microscopic determination of the wettability of similar surfactants on coal dust is provided, which is of great significance for the selection of surfactants for dust suppression by mist spraying in underground coal mines.

### BRIEF SUMMARY OF THE INVENTION

In view of the fact that the current selection of surfactants for mining is time-consuming and laborious, the present invention is intended to disclose a microscopic comparison and experimental verification method for the wettability of similar surfactants on coal dust, in order to greatly improve the efficiency of selecting surfactants with excellent wetting effect on coal dust and provide a scientific idea for more effectively reducing coal dust pollution.

In order to achieve the above objective, the present invention provides a microscopic comparison and experimental verification method for the wettability of similar surfactants on coal dust, comprising the following steps:
S1: collecting a coal sample on site from a mining area, crushing the coal sample into powder, and then analyzing the coal sample by infrared spectroscopy and nuclear magnetic resonance spectroscopy to obtain information on functional groups and carbon structure of coal molecules; and constructing three-dimensional macromolecular models of the coal sample and at least three similar surfactants respectively in combination with molecular design software ACD/ChemSketch and Materials Studio, wherein the similar surfactants refer to surfactants which are all anionic surfactants or are all nonionic surfactants;
S2: constructing a surfactant-coal molecule electrostatic interaction model and providing a principle of opposite potentials attract in combination with competitive adsorption, and analyzing an orbital energy difference and an electrostatic potential difference between surfactant molecules and coal molecules;
wherein the surfactant-coal molecule electrostatic interaction model involves a density functional theory, and B3LYP-D3 functional is selected with 6-311+G** as a basis set, and electrostatic potentials and orbital energy are visualized through Multiwfn+VMD; positive and negative electrostatic potential extremes of surfactant molecules are analyzed; if the negative or positive potential extreme of a surfactant molecule is less than that of a water molecule, interaction strength between the surfactant molecule and the water molecule is greater than interaction strength between water molecules, and water molecules are adsorbed near the surfactant molecule, thereby improving wettability; therefore, the greater the orbital energy difference and the electrostatic potential extreme, the better the wetting effect of a surfactant, and the better the wetting effect of the surfactant on coal dust;
S3: analyzing the strength and energy of hydrogen bonds between surfactant molecules and water molecules, wherein the strength is determined by the bond angle and bond length of the formed hydrogen bonds; and calculating average values of the bond length, bond angle and hydrogen bond energy of the hydrogen bonds formed between the surfactant molecules and the water molecules; the greater the strength of the hydrogen bonds formed between the surfactant molecules and the water molecules, the smaller the energy, the better the absorption effect on water molecules, and the better the wetting effect on the coal dust;
S4: constructing a surfactant-coal-water molecular periodic solution model, giving charges to the model through a COMPASSII force field, and then performing geometry optimization on the model to achieve a stable geometry;
S5: performing NPT simulation on the periodic solution model subjected to geometry optimization until the density of coal molecules, surfactant molecules and water molecules is consistent with experimental conditions, then performing NVT simulation, observing the movement of water molecules in different surfactant systems, calculating the number of hydrogen bonds formed between various surfactants in an equilibrium state and the water molecules and interaction energy between the surfactants and the water molecules,
wherein the calculation formula of the interaction energy is expressed as:

$${E}_{int ( surfactant - water )} = \frac{{E}_{total} - {E}_{water} - {E}_{{coal}_{+ surfactant}} - {E}_{surfactant} - {E}_{{coal}_{+ water}} + {E}_{coal} + {E}_{surfactant + water}}{2}$$

$${E}_{int ( coal - surfactant )} = \frac{{E}_{total} - {E}_{surfactant} - {E}_{{coal}_{+ water}} - {E}_{coal} - {E}_{surfactant + water} + {E}_{water} - {E}_{coal + surfactant}}{2}$$

E_{int(surfactant-water)} where is interaction energy between a surfactant and water in the system; E_{int(coal-surfactant)} is an interaction energy between coal and the surfactant in the system; Eₜₒₜₐₗ is a total energy of the system; E_{coal}, E_{water} and E_{surfactant} are the energy of coal, the energy of water and the energy of the surfactant, respectively; E_{coal+water}, E_{coal+surfactant} and E_{surfactant+water} are respectively total energies of two components in the system: the total energy of coal and water, the total energy of coal and the surfactant, the total energy of the surfactant and water; and the total energies of two components are calculated by removing a third component from the system, that is,

$${E}_{{coal}_{+ water}} = {E}_{total} - {E}_{surfactant} \cdot {E}_{coal + surfactant} = {E}_{total} - {E}_{water} , and {E}_{surfactant + water} = {E}_{total} - {E}_{coal} ;$$

the number of hydrogen bonds and interaction energy between various surfactants and water molecules are calculated; the more the hydrogen bonds formed between the surfactants and water molecules, the greater the interaction energy between the surfactants and water molecules; and
S6: selecting multiple concentration gradients within a concentration range of 0.01-0.14% to prepare various surfactant solutions, and defining the concentration of a surfactant when the surface tension reaches the minimum value as the optimal use concentration of the surfactant; pouring the weighed coal dust into a beaker containing the surfactant solution of the optimal concentration according to a ratio of 0.5 g coal powder to 250 mL surfactant solution of the optimal concentration, and recording the time the coal dust takes from coming into contact with the liquid surface to completely sinking into the liquid surface, the shorter the time, the better the wettability on coal, thereby verifying that the wettability under experimental conditions is consistent with the wettability of the surfactant on the coal dust obtained by the microscopic determination method from two perspectives: the orbital energy difference and electrostatic potential difference in quantum mechanics in step S2, and the number of hydrogen bonds and interaction energy in molecular dynamics in steps S3-S5.

As a preferred embodiment of the solution described, in step S2, in the process of constructing the surfactant-coal molecule electrostatic interaction model, one coal molecule and 50 water molecules; one surfactant molecule and 50 water molecules; one surfactant molecule and one coal molecule are selected.

More preferably, in step S4, the surfactant-coal-water molecular periodic solution model constructed comprises 10 coal molecules, 25 surfactant molecules and 2000 water molecules.

More preferably, in step S4, the surfactant-coal-water molecular periodic solution model needs to undergo geometry optimization-annealing treatment first so that the system energy reaches a lowest point and is in a stable state; during the process of geometry optimization, charges are given to different molecules by setting a COMPASSII force field, and a net charge of the system is 0.

More preferably, in step S5, after the periodic solution model is constructed, a force field and charges are firstly given to the periodic solution model, and 500 ps of NPT simulation is required to reach a density equilibrium state, thereby ensuring that the density of coal molecules, surfactants and water molecules has an error of less than 10% relative to an actual density of the coal sample and the density of water molecules is 1000 kg/m³; after the periodic solution model reaches density equilibrium, NVT simulation is performed to simulate the motion trajectories of different molecules in a confined space for a time of 1000 ps with a step size of 1 fs until the system reaches energy equilibrium.

More preferably, in step S1, the coal sample collected on site is crushed into powder by a ball mill.

The beneficial effects of the present invention are as follows:
(1) Determining the effect of surfactants in improving the wettability of solutions to coal dust from two independent microscopic perspectives, namely, the orbital energy difference and electrostatic potential difference in quantum mechanics and the number of hydrogen bonds and interaction energy in molecular dynamics, is more reliable than that from a single perspective.
(2) Since the coal sample is sampled on site and an accurate coal molecular model is constructed, on this basis, the determination for the wettability of a surfactant on coal is most suitable for this type of coal, with the characteristics of high pertinence and high accuracy;
(3) This method is performed for determination from two microscopic perspectives in quantum mechanics and molecular dynamics in combination with laboratory verification, and the conclusions obtained from the three aspects are completely consistent. In a subsequent practice, it is only necessary to select surfactants at the microscopic level from the perspective of quantum mechanics or molecular dynamics. The problems of long experimental time and high cost in current surfactant selection can be solved effectively, and a simple and effective determination method for the selection of surfactants is provided and has the advantages of short time, no cost and pre-selection function.

### BRIEF DESCRIPTION OF THE SEVERAL VIEWS OF THE DRAWINGS

FIG. 1 is a schematic flowchart of the present invention;

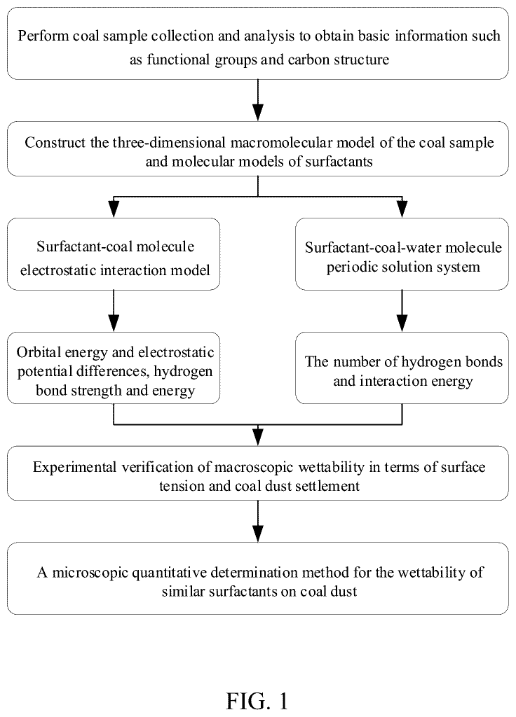

FIG. 2 shows three-dimensional macromolecular models of coal sample, SDBS, RPT and SDS; and

FIG. 3 shows the macroscopic wettability of SDBS, RPT and SDS on coal samples in terms of surface tension and settlement time.

### DETAILED DESCRIPTION OF THE INVENTION

To describe the present invention, the present invention will be further described in detail below with reference to the drawings and embodiments.

A microscopic comparison and experimental verification method for the wettability of similar surfactants on coal dust comprises the following steps.

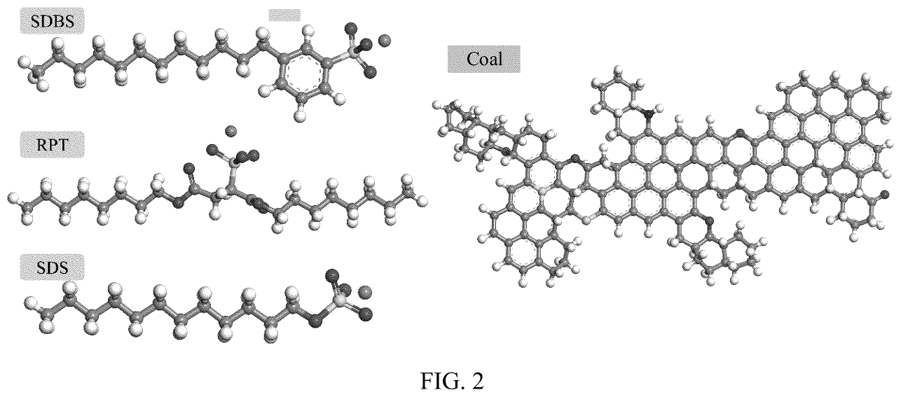

In step S1, a coal sample is collected on site from a mining area and crushed into powder; preferably, the coal sample is crushed by a ball mill; the coal sample is then analyzed by infrared spectroscopy and nuclear magnetic resonance spectroscopy to obtain information on the functional groups and carbon structure of coal molecules; and three-dimensional macromolecular models of the coal sample and at least three similar surfactants are constructed respectively in combination with molecular design software ACD/ChemSketch and Materials Studio, where the similar surfactants refer to surfactants which are all anionic surfactants or are all nonionic surfactants. Here, three anionic surfactants, i.e., SDBS, RPT and SDS, are used for demonstration, as shown in FIG. 2.

In step S2, a surfactant-coal molecule electrostatic interaction model is constructed, a principle of attraction of opposite potentials attract in combination with competitive adsorption is provided, and an orbital energy difference and an electrostatic potential difference between surfactant molecules and coal molecules are analyzed.

The surfactant-coal molecule electrostatic interaction model involves a density functional theory, and B3LYP-D3 functional is selected with 6-311+G** as a basis set, and electrostatic potential and orbital energy are visualized through Multiwfn+VMD; positive and negative electrostatic potential extremes of surfactant molecules are analyzed; if the negative or positive potential extreme of surfactant molecules is less than that of water molecules, interaction strength between surfactant molecules and water molecules is greater than interaction strength between water molecules, and water molecules are adsorbed near the surfactant molecules, thereby improving wettability; therefore, the greater the orbital energy difference and the electrostatic potential extreme, the better the wetting effect of a surfactant, and the better the wetting effect of the surfactant on coal dust.

Preferably, in step S2, in the process of constructing the surfactant-coal molecule electrostatic interaction model, one coal molecule and 50 water molecules; one surfactant molecule and 50 water molecules; one surfactant molecule and one coal molecule are selected.

The order of the surfactants in terms of electrostatic potential extremes and orbital energy difference is RPT, SDBS and SDS. It is preliminarily determined that the surfactant RPT has the best wetting effect on coal dust, while SDS has the worst. The results are shown in Table 1.

TABLE 1
Electrostatic potentials and molecular orbital
energy of surfactant-water-coal molecules

| | Positive | Negative | | |
|---|---|---|---|---|
| | potential | potential | LUMO | HOMO |
| | kcal/mol | kcal/mol | kcal/mol | kcal/mol |
| RPT | 24.53 | −49.93 | −18.81 | −150.48 |
| SDBS | 11.88 | −48.72 | −37.62 | −144.21 |
| SDS | 5.31 | −45.62 | −38.62 | −140.75 |
| water | 42.91 | −39.69 | — | — |

In step S3, the strength and energy of hydrogen bonds between surfactant molecules and water molecules are analyzed, where the strength is determined by the bond angle and bond length of the formed hydrogen bonds; and average values of the bond length, bond angle and hydrogen bond energy of the hydrogen bonds formed between the surfactant molecules and the water molecules are calculated; the greater the strength of the hydrogen bonds formed between the surfactant molecules and the water molecules, the smaller the energy, the better the absorption effect on water molecules, and the better the wetting effect on the coal dust. The results are shown in Table 2. The strength of the hydrogen bonds formed between RPT and water molecules is the greatest, the RPT has the smallest energy (negative value) with water molecules and has the best absorption effect on water molecules and the best wetting effect on coal dust.

TABLE 2
Average data of hydrogen bonds between
three surfactants and water molecules

| | Hydrogen bond | | |
|---|---|---|---|
| | potential | Bond length | Bond angle |
| Molecule | kcal/mol | Å | ° |
| RPT | −4.36 | 1.47 | 176.66 |
| SDBS | −2.81 | 1.61 | 154.01 |
| SDS | −2.74 | 2.17 | 150.81 |

In step S4, a surfactant-coal-water molecular periodic solution model is constructed, charges are given to the model through a COMPASSII force field, and then geometry optimization is performed on the model to achieve a stable geometry.

Preferably, in step S4, the surfactant-coal-water molecular periodic solution model constructed comprises 10 coal molecules, 25 surfactant molecules and 2000 water molecules.

Preferably, in step S4, the surfactant-coal-water molecular periodic solution model needs to undergo geometry optimization-annealing treatment first so that the system energy reaches a lowest point and is in a stable state; during the process of geometry optimization, charges are given to different molecules by setting the COMPASSII force field, and the net charge of the system is 0.

In step S5, NPT simulation is performed on the periodic solution model subjected to geometry optimization until the density of coal molecules, surfactant molecules and water molecules is consistent with experimental conditions, NVT simulation is then performed, the movement of water molecules in different surfactant systems are observed, and the number of hydrogen bonds formed between various surfactants in an equilibrium state and the water molecules and interaction energy between the surfactants and the water molecules are calculated.

The calculation formula of the interaction energy is expressed as:

$${E}_{int ( surfactant - water )} = \frac{{E}_{total} - {E}_{water} - {E}_{{coal}_{+ surfactant}} - {E}_{surfactant} - {E}_{{coal}_{+ water}} + {E}_{coal} + {E}_{surfactant + water}}{2}$$

$${E}_{int ( coal - surfactant )} = \frac{{E}_{total} - {E}_{surfactant} - {E}_{{coal}_{+ water}} - {E}_{coal} - {E}_{surfactant + water} + {E}_{water} - {E}_{coal + surfactant}}{2}$$

E_{int(surfactant-water)} where is interaction energy between a surfactant and water in the system; E_{int(coal-surfactant)} is an interaction energy between coal and the surfactant in the system; Eₜₒₜₐₗ is a total energy of the system; E_{coal}, E_{water} and E_{surfactant} are the energy of coal, the energy of water and the energy of the surfactant, respectively; E_{coal+water}, E_{coal+surfactant} and E_{surfactant+water} are respectively total energies of two components in the system: the total energy of coal and water, the total energy of coal and the surfactant, the total energy of the surfactant and water; and the total energies of two components are calculated by removing a third component from the system, that is, E_{coal+water}=Eₜₒₜₐₗ−E_{surfactant} (energy of coal and water in the system=total energy of the system−energy of the surfactant in the system),
E_{coal+surfactant}=Eₜₒₜₐₗ−E_{water} (energy of coal and the surfactant in the system=total energy of the system-energy of water in the system), and
E_{surfactant+water}=Eₜₒₜₐₗ−E_{coal} (energy of water and the surfactant in the system=total energy of the system-energy of coal in the system).

The number of hydrogen bonds and interaction energy between various surfactants and water molecules are calculated. The more the hydrogen bonds formed between the surfactants and water molecules, the greater the interaction energy between the surfactants and water molecules, the better the adsorption effect on water molecules during a dynamic adsorption process, and the better the wettability exhibited.

Preferably, in step S5, after the periodic solution model is constructed, a force field and charges are firstly given to the periodic solution model, and 500 ps of NPT simulation is required to reach a density equilibrium state, thereby ensuring that the density of coal molecules, surfactants and water molecules has an error of less than 10% relative to an actual density of the coal sample and the density of water molecules is 1000 kg/m³; after the periodic solution model reaches density equilibrium, NVT simulation is performed to simulate the motion trajectories of different molecules in a confined space for a time of 1000 ps with a step size of 1 fs until the system reaches energy equilibrium.

The number of hydrogen bonds formed and the interaction energy between the three surfactants and water molecules are shown in Table 3. RPT forms the largest number of hydrogen bonds with water molecules, has the greatest interaction energy with water molecules, has the best adsorption effect on water molecules during the dynamic adsorption process, and exhibits the best wettability.

TABLE 3
The number of hydrogen bonds formed and the interaction
energy between the three surfactants and water molecules

| | Number of | E_{int(S-W)} | E_{int(S-W)} |
|---|---|---|---|
| Molecule | hydrogen bonds | kcal/mol | kcal/mol |
| RPT | 43 | −5736.9 | −347.8 |
| SDBS | 37 | −4520.7 | −260.3 |
| SDS | 32 | −4252.2 | −175.4 |

In step S6, multiple concentration gradients are selected within a concentration range of 0.01-0.14% to prepare various surfactant solutions, and the concentration of a surfactant when the surface tension reaches the minimum value is defined as the optimal use concentration of the surfactant; the weighed coal dust is poured into a beaker containing the surfactant solution of the optimal concentration according to a ratio of 0.5 g coal powder to 250 mL surfactant solution of the optimal concentration, and the time the coal dust takes from coming into contact with the liquid surface to completely sinking into the liquid surface is recorded. The shorter the time, the better the wettability on coal. It is verified that the wettability under experimental conditions is consistent with the wettability of the surfactant on the coal dust obtained by the microscopic determination method from two perspectives: the orbital energy difference and electrostatic potential difference in quantum mechanics in step S2, and the number of hydrogen bonds and interaction energy in molecular dynamics in steps S3-S5.

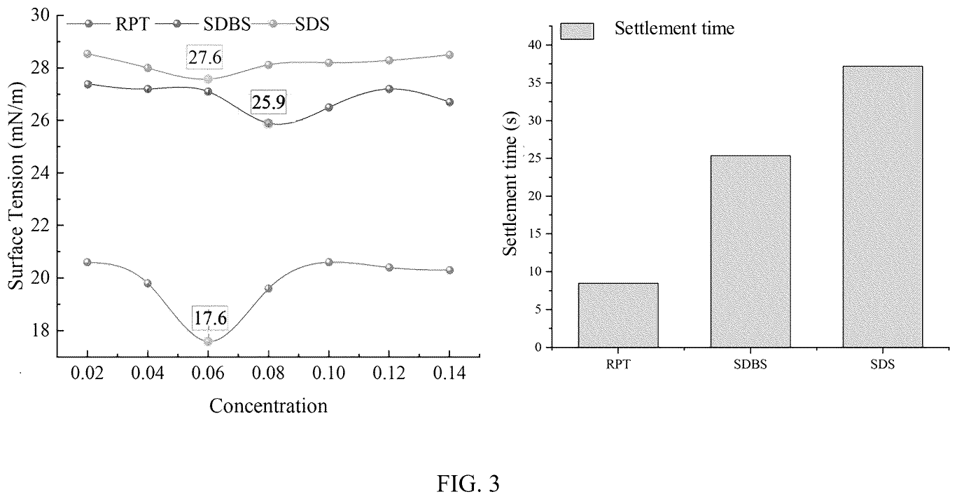

The experimental results of the three surfactants are shown in FIG. 3. RPT has the best wetting performance under the experimental conditions.

What is claimed is:

1. A microscopic comparison and experimental verification method for the wettability of similar surfactants on coal dust comprises the following steps:
S1: collecting a coal sample on site from a mining area, crushing the coal sample into powder, and then analyzing the coal sample by infrared spectroscopy and nuclear magnetic resonance spectroscopy to obtain information on functional groups and carbon structure molecules; and constructing three-dimensional macromolecular models of the coal sample and at least three similar surfactants respectively in combination with molecular design software ACD/ChemSketch and Materials Studio, wherein the similar surfactants refer to surfactants which are all anionic surfactants or are all nonionic surfactants;
S2: constructing a surfactant-coal molecule electrostatic interaction model and providing a principle of opposite potentials attract in combination with competitive adsorption, and analyzing an orbital energy difference and an electrostatic potential difference between surfactant molecules and coal molecules;
wherein the surfactant-coal molecule electrostatic interaction model involves a density functional theory, and B3LYP-D3 functional is selected with 6-311+G** as a basis set, and electrostatic potentials and orbital energy are visualized through Multiwfn+VMD; positive and negative electrostatic potential extremes of surfactant molecules are analyzed; if the negative or positive potential extreme of a surfactant molecule is less than that of a water molecule, interaction strength between the surfactant molecule and the water molecule is greater than interaction strength between water molecules, and water molecules are adsorbed near the surfactant molecule, thereby improving wettability; therefore, the greater the orbital energy difference and the electrostatic potential extreme, the better the wetting effect of a surfactant, and the better the wetting effect of the surfactant on coal dust;
S3: analyzing the strength and energy of hydrogen bonds between surfactant molecules and water molecules, wherein the strength is determined by the bond angle and bond length of the formed hydrogen bonds; and calculating average values of the bond length, bond angle and hydrogen bond energy of the hydrogen bonds formed between the surfactant molecules and the water molecules; the greater the strength of the hydrogen bonds formed between the surfactant molecules and the water molecules, the smaller the energy, the better the absorption effect on water molecules, and the better the wetting effect on the coal dust;
S4: constructing a surfactant-coal-water molecular periodic solution model, giving charges to the model through a COMPASSII force field, and then performing geometry optimization on the model to achieve a stable geometry;
S5: performing NPT simulation on the periodic solution model subjected to geometry optimization until the density of coal molecules, surfactant molecules and water molecules is consistent with experimental conditions, then performing NVT simulation, observing the movement of water molecules in different surfactant systems, and calculating the number of hydrogen bonds formed between various surfactants in an equilibrium state and the water molecules and interaction energy between the surfactants and the water molecules, wherein
a calculation formula of the interaction energy is expressed as:
$${E}_{int ( surfactant - water )} = \frac{{E}_{total} - {E}_{water} - {E}_{{coal}_{+ surfactant}} - {E}_{surfactant} - {E}_{{coal}_{+ water}} + {E}_{coal} + {E}_{surfactant + water}}{2}$$
$${E}_{int ( coal - surfactant )} = \frac{{E}_{total} - {E}_{surfactant} - {E}_{{coal}_{+ water}} - {E}_{coal} - {E}_{surfactant + water} + {E}_{water} - {E}_{coal + surfactant}}{2}$$
E_{int(surfactant-water)} where is interaction energy between a surfactant and water in the system; E_{int(coal-surfactant)} is an interaction energy between coal and the surfactant in the system; Eₜₒₜₐₗ is a total energy of the system; E_{coal}, E_{water} and E_{surfactant} are the energy of coal, the energy of water and the energy of the surfactant, respectively; E_{coal+water}, E_{coal+surfactant} and E_{surfactant+water} are respectively total energies of two components in the system: the total energy of coal and water, the total energy of coal and the surfactant, the total energy of the surfactant and water; and the total energies of two components are calculated by removing a third component from the system, that is, E_{coal+water}=Eₜₒₜₐₗ−E_{surfactant}·E_{coal+surfactant}=Eₜₒₜₐₗ−E_{water}, and E_{surfactant+water}=Eₜₒₜₐₗ−E_{coal};
the number of hydrogen bonds and interaction energy between various surfactants and water molecules are calculated; the more the hydrogen bonds formed between the surfactants and water molecules, the greater the interaction energy between the surfactants and water molecules, the better the adsorption effect on water molecules during a dynamic adsorption process, and the better the wettability exhibited; and
S6: selecting multiple concentration gradients within a concentration range of 0.01-0.14% to prepare various surfactant solutions, and defining the concentration of a surfactant when the surface tension reaches the minimum value as the optimal use concentration of the surfactant; pouring the weighed coal dust into a beaker containing the surfactant solution of the optimal concentration according to a ratio of 0.5 g coal powder to 250 mL surfactant solution of the optimal concentration, and recording the time the coal dust takes from coming into contact with the liquid surface to completely sinking into the liquid surface, the shorter the time, the better the wettability on coal, thereby verifying that the wettability under experimental conditions is consistent with the wettability of the surfactant on the coal dust obtained by the microscopic determination method from two perspectives: the orbital energy difference and electrostatic potential difference in quantum mechanics in step S2, and the number of hydrogen bonds and interaction energy in molecular dynamics in steps S3-S5.

2. The microscopic comparison and experimental verification method for the wettability of similar surfactants on coal dust according to claim 1, wherein in step S2, in the process of constructing the surfactant-coal molecule electrostatic interaction model, one coal molecule and 50 water molecules; one surfactant molecule and 50 water molecules; one surfactant molecule and one coal molecule are selected.

3. The microscopic comparison and experimental verification method for the wettability of similar surfactants on coal dust according to claim 1, wherein in step S4, the surfactant-coal-water molecular periodic solution model constructed comprises 10 coal molecules, 25 surfactant molecules and 2000 water molecules.

4. The microscopic comparison and experimental verification method for the wettability of similar surfactants on coal dust according to claim 1, wherein in step S4, the surfactant-coal-water molecular periodic solution model needs to undergo geometry optimization-annealing treatment first so that the system energy reaches a lowest point and is in a stable state; during the process of geometry optimization, charges are given to different molecules by setting a COMPASSII force field, and a net charge of the system is 0.

5. The microscopic comparison and experimental verification method for the wettability of similar surfactants on coal dust according to claim 1, wherein in step S5, after the periodic solution model is constructed, a force field and charges are firstly given to the periodic solution model, and 500 ps of NPT simulation is required to reach a density equilibrium state, thereby ensuring that the density of coal molecules, surfactants and water molecules has an error of less than 10% relative to an actual density of the coal sample and the density of water molecules is 1000 kg/m³; after the periodic solution model reaches density equilibrium, NVT simulation is performed to simulate the motion trajectories of different molecules in a confined space for a time of 1000 ps with a step size of 1 fs until the system reaches energy equilibrium.

6. The microscopic comparison and experimental verification method for the wettability of similar surfactants on coal dust according to claim 1, wherein in step S1, the coal sample collected on site is crushed into powder by a ball mill.
